(19) United States
(12) Patent Application Publication
(10) Pub. No.: US 2021/0010936 A1
(43) Pub. Date: Jan. 14, 2021

(54) CHARACTERIZING DIESEL CONTAMINATED WITH HYDROCARBONS

(71) Applicant: Saudi Arabian Oil Company, Dhahran (SA)

(72) Inventors: Ezzat Hegazi, Windsor (CA); Vincent Cunningham, Ferbane (IE)

(21) Appl. No.: 16/509,046

(22) Filed: Jul. 11, 2019

(51) Int. Cl. G01N 21/64

(52) U.S. Cl. CPC G01N 21/6402; G01N 21/6456; G01N 2021/6463; G01N 2201/0484; G01N 2201/06113; G01N 2201/12753; G01N 2021/6417

(57) ABSTRACT

To characterize hydrocarbon contamination, a container, an ultraviolet laser source and a detector are spatially positioned relative to each other. The container carries a hydrocarbon sample including a first hydrocarbon and a second hydrocarbon. The ultraviolet laser source is configured to emit an ultraviolet laser at a wavelength to irradiate the hydrocarbon sample in the container. The wavelength is configured to induce fluorescence in the hydrocarbon sample. The detector is configured to detect the induced fluorescence. The hydrocarbon sample in the container is irradiated with the ultraviolet laser at multiple locations within the container at respective multiple distances from the detector. The multiple locations are arranged in a straight line normal to the detector. A volume of the first hydrocarbon in the hydrocarbon sample is determined based on induced fluorescence detected by the detector at each of the multiple locations arranged in the straight line normal to the detector

### TECHNICAL FIELD

This disclosure relates to analyzing hydrocarbons using fluorescent spectroscopy.

### BACKGROUND

Fluorescent spectroscopy can be used for the qualitative and quantitative analysis of gases, liquids and solids that exhibit the phenomena of fluorescence and phosphorescence. When molecules are irradiated by energy of a particular frequency or wavelength, the electrons experience a transition from the ground state to an excited state due to the absorbance of photons. The electrons return to the ground state by any of several different routes known as deactivation processes. The preferred route is the path that provides the shortest lifetime in the excited state. For certain compounds under appropriate conditions, fluorescence is the preferred deactivation process. Generally, a molecule excited at an absorption frequency will exhibit fluorescence at a lower frequency—longer wavelength emission band. Fluorescent spectroscopy can be used to analyze hydrocarbons, for example, hydrocarbons produced from subsurface reservoirs in which the hydrocarbons are entrapped. In some instances, this technique can be implemented to determine if a hydrocarbon is mixed with one or more other hydrocarbons.

### SUMMARY

This disclosure describes techniques relating to characterizing diesel contaminated with hydrocarbons. In particular, this disclosure describes a depth-specific fluorescence technique to characterize hydrocarbon samples and quantify contaminant volumes in hydrocarbon samples.

Certain aspects of the subject matter described here can be implemented as a method. A container, an ultraviolet laser source and a detector are spatially positioned relative to each other. The container carries a hydrocarbon sample including a first hydrocarbon and a second hydrocarbon. The ultraviolet laser source is configured to emit an ultraviolet laser at a wavelength to irradiate the hydrocarbon sample in the container. The wavelength is configured to induce fluorescence in the hydrocarbon sample. The detector is configured to detect the induced fluorescence. The hydrocarbon sample in the container is irradiated with the ultraviolet laser at multiple locations within the container at respective multiple distances from the detector. The multiple locations are arranged in a straight line normal to the detector. A volume of the first hydrocarbon in the hydrocarbon sample is determined based on induced fluorescence detected by the detector at each of the multiple locations arranged in the straight line normal to the detector.

An aspect, combinable with any of the other aspects, can include the following features. The induced fluorescence includes a plot of fluorescence intensity over a range of wavelengths. The plot of the fluorescence over the range of wavelengths at each of the multiple locations includes a greatest fluorescence intensity at a first wavelength of the range of wavelengths and a second greatest fluorescence intensity at a second wavelength of the range of wavelengths. To determine the volume of the first hydrocarbon, a respective greatest fluorescence intensity and a second greatest fluorescence intensity over the range of wavelengths is determined at each of the multiple locations. A fluorescence intensity ratio of the greatest and second greatest fluorescence intensity ratios is determined at each of the multiple locations.

An aspect, combinable with any of the other aspects, can include the following features. To determine the volume of the first hydrocarbon, a calibration plot of the multiple locations to multiple known volume ratios is plotted. Each known volume ratio is a ratio of a known volume of the first hydrocarbon to a known volume of the hydrocarbon sample.

An aspect, combinable with any of the other aspects, can include the following features. The multiple known volume ratios includes six volume ratios.

An aspect, combinable with any of the other aspects, can include the following features. The six volume ratios are 100%:0%, 95%:5%, 90%:10%, 85%:15%, 80%:20% and 50%:50%.

An aspect, combinable with any of the other aspects, can include the following features. To construct the calibration plot, each known hydrocarbon sample is prepared by mixing a known volume of the first hydrocarbon with a known volume of the hydrocarbon sample resulting in a plurality of known hydrocarbon samples. For each known hydrocarbon sample, a quantity of each known hydrocarbon sample is plated in the container and irradiated with the ultraviolet laser at the multiple locations within the container at the respective multiple distances from the detector.

An aspect, combinable with any of the other aspects, can include the following features. To construct the calibration plot, for each known hydrocarbon sample, a respective greatest and second greatest fluorescence intensities over the range of wavelengths is measured at each of the multiple locations. A fluorescence intensity ratio of the greatest and second greatest fluorescence intensity ratios is determined at each of the multiple locations resulting in multiple fluorescence intensity ratios at the multiple respective locations for each known hydrocarbon sample.

An aspect, combinable with any of the other aspects, can include the following features. To construct the calibration plot, a plot of the multiple fluorescence intensity ratios at the multiple respective locations for the multiple known hydrocarbon samples is constructed. A known fluorescence intensity ratio that is the same at the multiple locations for the multiple known hydrocarbon samples is identified as a reference fluorescence intensity ratio.

An aspect, combinable with any of the other aspects, can include the following features. To determine the volume of the first hydrocarbon, from the calibration plot, a location at which the fluorescence intensity ratio matches the reference fluorescence intensity ratio is determined.

Certain aspects of the subject matter described here can be implemented as a system. The system includes a container configured to carry a hydrocarbon sample including a first hydrocarbon and a second hydrocarbon. The system includes an ultraviolet laser source configured to emit an ultraviolet laser at a wavelength to irradiate the hydrocarbon sample in the container. The wavelength induces fluorescence in the hydrocarbon sample. The system includes a detector configured to detect the fluorescence. The ultraviolet laser source, is positioned spatially relative to the container and the detector to irradiate the hydrocarbon sample in the container with the ultraviolet laser at multiple locations within the container at multiple respective distances from the detector. The multiple locations are arranged in a straight line normal to the detector. The system includes a computer system which includes one or more processors and a computer-readable medium. The medium stores instructions executable by the one or more processors to perform operations described here.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a schematic diagram of an example of a laser induced fluorescence (LIF) measurement system.

FIGS. 2A-2E are schematic diagrams of examples of measuring LIF intensities in a hydrocarbon sample using the system of FIG. 1.

FIG. 3 is a flowchart of an example of a process of operating the system of FIG. 1.

FIG. 4A shows LIF spectra of five hydrocarbon samples having different percentage volumes.

FIG. 4B shows LIF spectra of a first hydrocarbon sample measured at five different depths.

FIG. 4C shows LIF spectra of a second hydrocarbon sample measured at five different depths.

FIG. 4D shows LIF spectra of a third hydrocarbon sample measured at five different depths.

FIG. 5 is a plot of fluorescence intensity ratios versus depths generated from multiple LIF spectra of multiple hydrocarbon samples.

FIG. 6 is a calibration plot generated from the plot of FIG. 5.

Like reference numbers and designations in the various drawings indicate like elements.

### DETAILED DESCRIPTION

This disclosure describes a LIF process implemented to identify contamination in a hydrocarbon sample, for example, a refined diesel mixed with other hydrocarbons. In some implementations, a light source (for example, a laser source), a container carrying the hydrocarbon sample and a detector are spatially arranged as described later. Light from the light source irradiates the hydrocarbon sample in the container causing the hydrocarbon sample to fluoresce. The detector measures the LIF intensity. A depth of the container relative to the detector, that is, a distance between the container and the detector, is changed, and the LIF intensity is measured. The steps of measuring the LIF intensity is repeated for different depths of the container relative to the detector. As described later, fluorescence intensity ratios are determined for each depth, and a plot of fluorescence intensity ratios versus depths is constructed. A calibration plot of depths versus percentage volumes of hydrocarbons in the hydrocarbon sample is generated from the plot of fluorescence intensity ratios versus depths. Using the calibration plot, an unknown volume of a hydrocarbon in an unknown hydrocarbon sample can be determined.

The techniques described in disclosure can be implemented to measure contamination in hydrocarbon samples that includes two different hydrocarbons in unknown volumes. The systematic changes in the spectral profiles (that is, fluorescence intensity v/s wavelength) with respect to the depth at which the LIF is induced can be used in a comparative analysis to both characterize hydrocarbon samples and identify hydrocarbon-hydrocarbon contaminations. The techniques can further be implemented for purity testing the presence of a pure hydrocarbon, presence and concentration of hydrocarbon contaminants, presence of certain types of gasoline or their mixtures, and generally for testing any type of liquids that exhibit fluorescence (not only hydrocarbons).

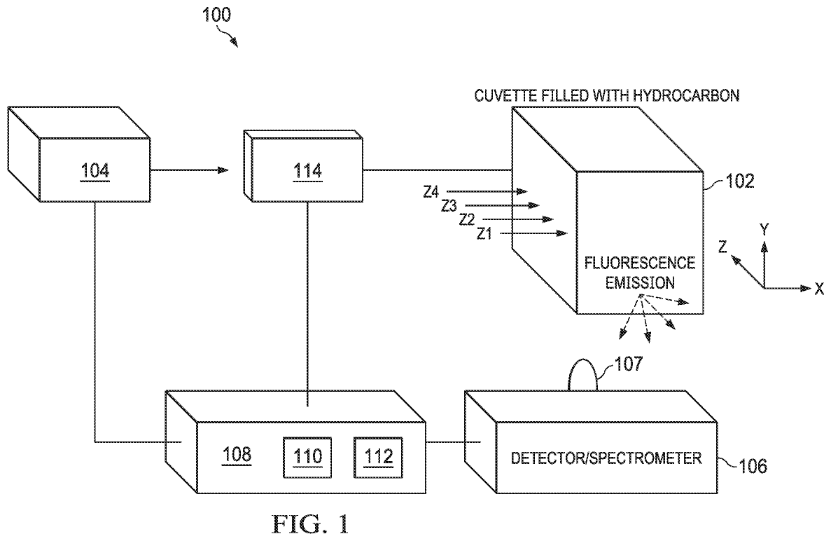

FIG. 1 is a schematic diagram of an example of a laser induced fluorescence (LIF) measurement system 100. The system 100 can be implemented to characterize hydrocarbon samples by a comparative analysis of fluorescence spectra at different geometrical positions within the sample. A hydrocarbon sample is either a pure hydrocarbon or a mixture of two hydrocarbons, one of which is a contaminant. In some examples, the contaminant can be a refined diesel contained in or mixed with a different hydrocarbon family to a volume as low as 5% volume of hydrocarbon per volume of solution (% v/v). When a fluorescing hydrocarbon sample is irradiated with UV radiation at an excitation wavelength, the sample emits light at a wavelength longer than the excitation wavelength. The emitted fluorescence is recorded as a fluorescence spectrum that includes a shape and has characteristics such as a spectral region and temporal behavior that depend on the volume of each hydrocarbon in the hydrocarbon sample. As described later, the system 100 can be implemented to characterize the hydrocarbon samples based on the variation in the fluorescence spectra of the same sample at different depths (that is, distances from a fluorescence detector) and of different samples, each having a different volume of a first hydrocarbon in a volume of a hydrocarbon sample made up of two hydrocarbons. For example, the % v/v values of the multiple hydrocarbon samples can be 0% (that is, 0% volume of the first hydrocarbon and 100% volume of the second hydrocarbon), 5% (that is, 5% volume of the first hydrocarbon and 95% volume of the second hydrocarbon), 10% (that is, 10% volume of the first hydrocarbon and 90% volume of the second hydrocarbon), 15% (that is, 15% volume of the first hydrocarbon and 85% volume of the second hydrocarbon), 20% (that is, 20% volume of the first hydrocarbon and 80% volume of the second hydrocarbon) and 50% (that is, 50% volume of the first hydrocarbon and 50% volume of the second hydrocarbon).

Returning to FIG. 1, the system 100 includes a container 102 configured to carry a hydrocarbon sample that can include two hydrocarbons. In some implementations, the container 102 can be, for example, a standard cuvette, test tube or other container, of dimensions sufficient to carry a pre-determined volume of the hydrocarbon sample. For example, the container 102 can have dimensions of 10 millimeter (mm)×10 mm×40 mm or 10 mm×10 mm×50 mm. The container 102 can be made of a material that is optically transparent and chemically inert. For example, the container 102 can be made using quartz, fused silica or similar material having a high transmittance to ultraviolet radiation.

The system 100 includes an ultraviolet laser source 104 configured to emit a laser, for example, an ultraviolet laser, at a wavelength to irradiate the hydrocarbon sample in the container 102. The wavelength can induce fluorescence in the hydrocarbon sample. For example, the wavelength range can be from UV to part of the visible spectrum, that is, 225 nanometers (nm) to 532 nm. The specific wavelength used with a sample will depend on the mixtures being tested. For example, to detect diesel at 380 nm wavelength, the laser wavelength can be as low as 225 nm. For heavier hydrocarbons, a wavelength of 450 nm can be implemented. In some implementations, the ultraviolet laser source 104 is a Nd:YAG laser which can emit a pulsed laser having a pulse of approximately 6-8 nanoseconds (ns) and energy of about 30-50 milliJoules (mJ) per pulse. In some implementations, the laser beam has a wavelength of 349 nanometers (nm). Optical filters can be positioned, for example, in front of the Nd:YAG laser head to filter infrared and green radiations that may originate from the laser source 104.

The laser source 104 generates a laser beam which penetrates the container 102 to irradiate the hydrocarbon sample within. In some implementations, the laser beam can be transmitted directly to the container 102. In some implementations, the laser beam can be diverted, for example, through optical equipment like mirrors or lenses or both being transmitted to the container 102. In some implementations, to control the size of the laser beam that impinges on the hydrocarbon sample in the container 102, an adjustable iris can be placed in the path of the laser beam before the laser beam enters the container 102. Alternatively or in addition, steering optics (for example, an iris, mirrors, optical fibers, lenses, any combination of them or other optical equipment) can be used to control a size of the laser beam that impinges upon the hydrocarbon sample in the container 102 as well as the location within the container 102 at which the laser beam contacts the hydrocarbon sample.

The system 100 includes a detector 106 configured to detect the fluorescence induced when the laser from the laser source 104 irradiates the hydrocarbon sample in the container 102. The detector 106 can include a spectrometer with a charge couple device (CCD). The CCD can have a fast time response and a resolution of about 1.5 ns. The detector 106 can convert the induced fluorescence into a signal (for example, a voltage signal). In some implementations, steering optics 107 can be used to maximize the induced fluorescence captured by the detector 106. For example, the steering optics 107 can include one or more optical filters that can filter out light in the wavelength outside that of the induced fluorescence.

Alternatively or in addition, an ICCD camera can be used as the detector 106. In some implementations, the seeing the spectrum can allow determining peaks of interest to determine best ratios (described later). Once the best peaks for the ratios have been determined, a simple optical detector (not a spectrometer) can be used, for example, with a filter for the different optical peaks being detected. In such situations, the total average intensity of the fluorescing light is obtained over the bandwidth of the filter. Further, filtering the wavelength of the light source from the detector 106 will avoid saturation due to the intensity of the light source.

The system 100 includes a computer system 108 which includes one or more processors 110 and a computer-readable medium 112 storing instructions executable by the one or more processors 110. The computer system 108 can be operatively coupled (for example, using wired or wireless techniques) to one or more or all components of the system 100. For example, the computer system 108 can receive voltage signals from the detector 106, the voltage signals representing the induced fluorescence captured by the detector. In another example, the computer system 108 can send control signals to turn the laser source 104 on or off. In some implementations, the computer system 108 can analyze the data received from the detector 106 to analyze or characterize (or both) the hydrocarbon sample in the container 102, as explained later.

In some implementations, the laser source 104, the container 102 and the detector 106 can be spatially arranged relative to each other in a 90° orientation. For example, if the laser source 104 and the container 102 are arranged in an X-axis of a Cartesian coordinate system, the container 102 and the detector 106 are arranged in the Z-axis. In such an arrangement, the height of the container 102 is aligned with the Y-axis and originates, for example, at the bottom of the container 102. As described later, when inducing fluorescence in a hydrocarbon sample carried in the container 102, the distance between the laser source 104 and the container 102 (that is, distance along X-axis) and the distance from the bottom of the container 102 at which the laser beam impinges the hydrocarbon sample (that is, distance along Y-axis) remain constant. The distance between the location in the container 102 in which the laser beam impinges the hydrocarbon sample and the detector 106 (that is, distance along Z-axis, sometimes referred to as “depth”) is variable.

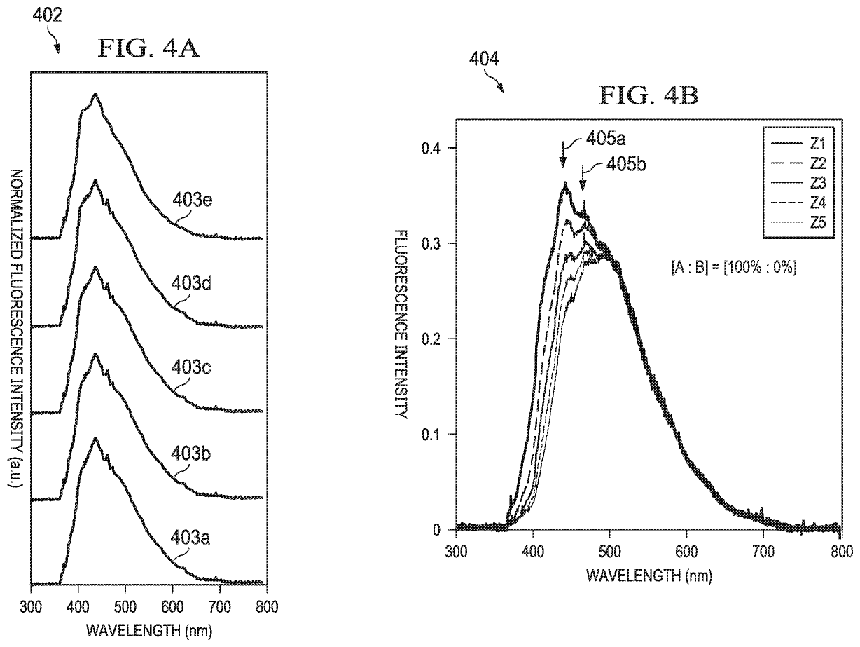

FIG. 4A shows LIF spectra 402 of five hydrocarbon samples having different percentage volumes measured by spatially arranging the laser source 104, the container 102 and the detector 106 in the 90° orientation. In FIG. 4A, the X-axis is the wavelength (nm) at which a hydrocarbon sample emits fluorescence and the Y-axis is the normalized fluorescence intensity (unitless). One of the five hydrocarbon samples is lubrication oil without a second hydrocarbon (Sample 1—100% lubrication oil, 0% second hydrocarbon). The remaining four hydrocarbon samples have varying volumes of lubrication oil and refined diesel: Sample 2—95% lubrication oil, 5% refined diesel; Sample 3—90% lubrication oil, 10% refined diesel; Sample 5—80% lubrication oil, 20% refined diesel; Sample 6—50% lubrication oil, 50% refined diesel. The LIF spectra 402 excludes that of Sample 4, which includes 85% lubrication oil and 15% refined diesel. The fluorescence spectrum of Sample 1, Sample 2, Sample 3, Sample 5 and Sample 6 are shown in FIG. 4A as plot 403a, plot 403b, plot 403c, plot 403d and plot 403e.

Each sample has a maximum fluorescence intensity (that is, peak intensity) close to a wavelength of 440 nm. To ease observation of the difference, the intensity of each plot in FIG. 4A has been normalized at the wavelength of 440 nm. Between the five samples, the shapes of the fluorescence spectra are not significantly different. However, as the volume of the refined diesel in the sample increases, a difference to the fluorescence intensity at a wavelength less than 440 nm becomes visible. In particular, the fluorescence intensities for a band of wavelengths less than 440 nm has a smaller difference for Sample 5 (with 50% refined diesel). In comparison, the corresponding fluorescence intensities for the same band of wavelengths has a larger difference for Sample 1 (with 0% refined diesel). The differences between the corresponding fluorescence intensities for each of Sample 2, Sample 3 and Sample 4 fall between the differences for Sample 1 and Sample 5. As described later, the differences between the fluorescence intensities in the band of wavelengths less than the wavelength at which the peak intensity is measured (440 nm, in this example) are used to characterize the hydrocarbon samples. In addition, for each of the five samples, by modifying the distances between the location in the container 102 in which the laser beam impinges the hydrocarbon sample and the detector 106, the volume of one hydrocarbon in the hydrocarbon sample can be determined to percentages as low as 5% v/v.

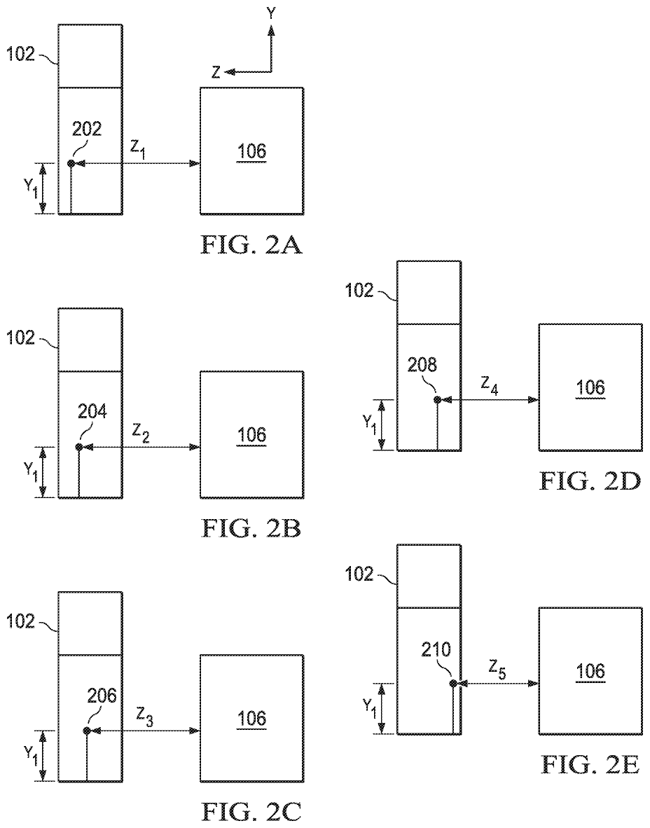

FIGS. 2A-2E are schematic diagrams of examples of measuring LIF intensities in a hydrocarbon sample using the system of FIG. 1. Each schematic diagram shows LIF intensity measurement at a respective depth that is different from other depths. At each depth, the hydrocarbon sample is positioned at a respective distance from the laser source 104. The multiple locations of the hydrocarbon sample are in a straight line normal to the detector, that is, to the surface of the detector that senses the fluorescence. The changes in depths can be implemented by a motor 114, for example, a stepper motor, included in the system 100. The laser source 104 and the container 102 can be positioned on a stage (not shown) that is substantially horizontal in the XZ plane. The motor 114 can operate to move the stage, in steps, along the Z-axis to different depths between the container 102 and the detector 106.

In each of the schematic diagrams shown in FIGS. 2A-2E, the laser source 104 is spatially positioned to irradiate the container 104 at a location within the container 102. At each location, the detector 106 measures the LIF. The distance from the origin along the Y-axis (Y1) and that along the X-axis (X1, not shown) remain constant for all LIF measurements. The distance between the detector 106 and the location within the container 102 varies for each measurement. For example, the location 202 (FIG. 2A), the location 204 (FIG. 2B), the location 206 (FIG. 2C), the location 208 (FIG. 2D) and the location 210 (FIG. 2D) are at distances (or depths) of Z₁, Z₂, Z₃, Z₄ and Z₅, respectively, each distance different from the other. In some implementations, as described earlier, the detector 106 can be at a fixed location and the stage supporting the container 102 and the laser 104 can be moved to different depths, for example, using the motor 114. Alternatively, the stage can be at the fixed location and the detector 106 can be moved to different depths. The computer system 108 can transmit instructions to the motor 114 to step-wise vary the depths. At each depth, the location of the laser remains within the container 102 and at the same height relative to the origin of the Y-axis, for example, the bottom of the container 102.

As described in this disclosure, the distance between the laser source 104 and the container 102 remains fixed and the distance between the container 102 and the detector 106 varies. Doing so causes the intensity of the short-wavelength part of the broad spectrum to drop faster than that of the long-wavelength part. This variation in the rate at which the intensity drops is a consequence of the different broad spectrum structures of different hydrocarbons and is used to detect the presence of different hydrocarbons in a hydrocarbon sample. In the Z-axis, the amount of fluorescing light traveling through the liquid before reaching the detector 106 changes affecting the ratio of the peaks. The optical attenuation (whether due to scattering or absorption) affects different wavelengths in different ways, allowing the ratio between the peaks to be determined and the sample to be characterized. Varying the distance along the X-axis is an alternative or additional mechanism to determine the concentration. With such methods, it is the intensity of the optical source, not the fluorescence, that will be attenuated. As the source penetrates the sample, it will be attenuated (scattered or absorbed or both) creating different intensities at different points as the light enters the sample. In contrast, as described in this disclosure, the fluorescing light in this case passes through the sample to reach the detector; thus, attenuation is a constant for the fluorescing light. In sum, as described in this disclosure, the light source is not traveling longitudinally through the sample; consequently, there is no attenuation effect. However, the light source itself is impinging onto the sample at different depths creating a scattering variation that modifies the measured fluorescence spectrum. The short wavelength part of the spectrum drops in intensity much more than the longer part as the light source moves away from the detection edge of the sample. This drastic contrast allows the technique to be used in the detection of certain types of hydrocarbons even if they are hidden inside other types.

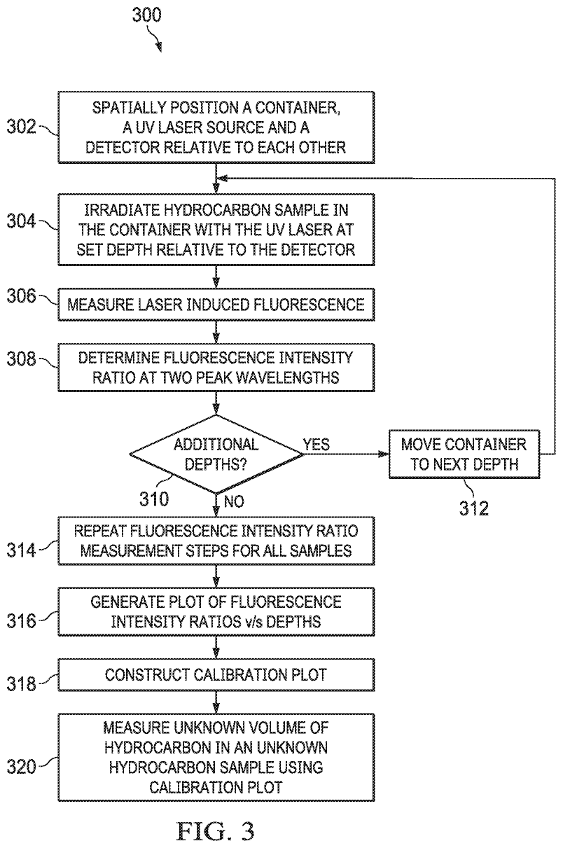

FIG. 3 is a flowchart of an example of a process 300 of operating the system of FIG. 1. The process 300 can be implemented by the ultraviolet laser source 104, the detector 106 and the computer system 108 in conjunction with a human operator. At 302, the container 102, the laser source 104 and the detector 106 can be positioned spatially relative to each other. For example, the human operator can spatially arrange the laser source 104, the detector 106 and the computer system 108 in the 90° orientation described earlier.

At 304, the hydrocarbon sample in the container 102 is irradiated with the laser from the laser source 104 at a set depth relative to the detector 106. For example, the computer system 108 controls the motor 114 to position the container 102 at a depth Z₁ from the detector 106. The laser source 104 transmits the laser into the hydrocarbon sample in the container 102. The hydrocarbon sample fluoresces responsive to being irradiated by the laser.

At 306, laser induced fluorescence is measured. For example, the detector 106 measures the laser induced fluorescence at the depth Z₁. At 308, a fluorescence intensity ratio at two peak wavelengths is determined. As described earlier, the induced fluorescence can be presented as a plot of fluorescence intensities over a range of wavelengths. The fluorescence intensity ratio is a ratio of the greatest fluorescence intensity at a first wavelength and a second greatest fluorescence intensity at a second wavelength of the range of wavelengths. That is, if the fluorescence intensities in the spectra were ranked (for example, from largest to smallest or vice versa), the fluorescence intensity ratio is the ratio of the largest intensity value to the second-to-largest intensity value. The wavelengths at which the top two fluorescence intensity values are measured are sometimes called the peak wavelengths. For example, the computer system 108 is configured to receive the fluorescence measured by the detector 106 over the range of wavelengths at depth Z₁, rank the fluorescence intensity values, identify the largest and second-to-largest intensity values, and determine the fluorescence intensity ratio. In this manner, the computer system 108 determines the fluorescence intensity ratio at one depth (Z₁) for a first hydrocarbon sample (for example, Sample 1).

At 310, a check is made to determine if the fluorescence intensity ratio is to be determined at additional depths. For example, the check can be implemented by the human operator or by the computer system 108 or both. If additional fluorescence intensity ratios are to be determined (decision branch “Yes”), then, at 312, the stage is moved to the next depth. For example, the human operator or the computer system 108 can operate the motor 114 to change the distance between the container 102 and the detector 106 to Z₂ (or one of the other depths). Steps 304, 306 and 308 are repeated at the depth. The check at step 310 is repeated and, if needed, Step 312 are repeated until, for the hydrocarbon sample, a fluorescence intensity ratio is determined for each depth. For example, for Sample 1, the steps 304, 306, 308 and 310 are repeated at each of Z₁, Z₂, Z₃, Z₄ and Z₅. As the depth from the detector 106 increases, the rate at which the greatest fluorescence intensity, which is observed at a lower wavelength, decreases is different from the rate at which the second-to-greatest fluorescence intensity, which is observed at a higher wavelength, decreases. This phenomenon is due to the Rayleigh scattering effect according to which the scattering intensity is inversely proportional to the fourth power of the wavelength. Therefore, the intensity at the shorter wavelength drops faster than the intensity at the higher wavelength. FIG. 4B shows LIF spectra 404 of a first hydrocarbon sample (Sample 1) measured at five different depths (Z₁, Z₂, Z₃, Z₄ and Z₅). In FIG. 4B, ref. nos. 405a and 405b identify the two wavelengths at which the fluorescence intensity values are the greatest and second-to-greatest. Unlike FIG. 4A, the fluorescence intensity values in FIG. 4B have not been normalized to the wavelength with the greatest fluorescence intensity value.

At 314, the steps to determine fluorescence intensity ratio measurements are repeated for all samples. For example, for each of Sample 2, Sample 3, Sample 4 and Sample 5, steps 306, 308, 310 and 312 (as needed) are repeated. FIG. 4C shows LIF spectra 406 of a second hydrocarbon sample (Sample 2) measured at five different depths (Z₁, Z₂, Z₃, Z₄ and Z₅). In FIG. 4C, ref. nos. 407a and 407b identify the two wavelengths at which the fluorescence intensity values are the greatest and second-to-greatest. FIG. 4D shows LIF spectra 408 of a third hydrocarbon sample (Sample 5) measured at five different depths (Z₁, Z₂, Z₃, Z₄ and Z₅). In FIG. 4D, ref. nos. 409a and 409b identify the two wavelengths at which the fluorescence intensity values are the greatest and second-to-greatest.

In some implementations, the computer system 108 can store each fluorescence spectrum at each depth for each sample. The computer system 108 can display each fluorescence spectrum in a display device (not shown) operatively coupled to the computer system 108. In addition, for each fluorescence spectrum, the computer system 108 can store the greatest and second-to-greatest fluorescence intensity values, determine a ratio of the two and store the ratio. For the five samples made using lubrication oil and refined diesel, the wavelengths at which the greatest and second-to-greatest fluorescence intensities are measured is 440 nm and 465 nm, respectively. In this manner, the computer system 108 can determine and store, for each sample and at each depth, a corresponding fluorescence intensity ratio.

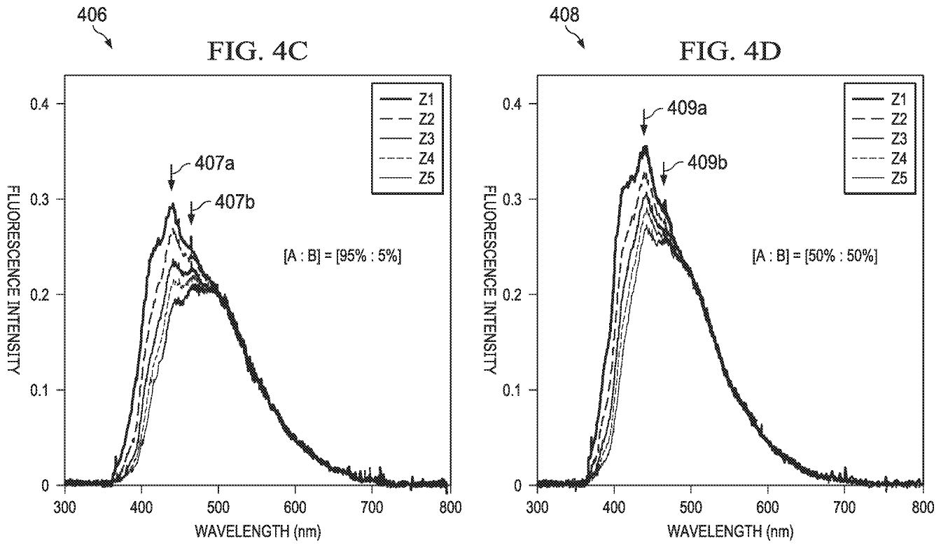

Returning to FIG. 3, at 316, a plot of fluorescence intensity ratios versus depths can be generated. For example, the computer system 108 can generate the plot using information determined and stored as described earlier. FIG. 5 is a plot 502 of fluorescence intensity ratios versus depths generated from multiple LIF spectra of multiple hydrocarbon samples. In plot 502, the X-axis is depths (that is, the distances between the container 102 and the detector 106) and the Y-axis is the fluorescence intensity ratio (unitless). The plot 502 includes six straight lines (lines 505a, 505b, 505c, 505d, 505e, 505f) fitted for the fluorescence intensity ratios determined for the six samples (Sample 1, Sample 2, Sample 3, Sample 4, Sample 5, Sample 6, respectively). For example, for Sample 1, the ratio of the greatest and second-to-greatest fluorescence intensities decreases as the depth, that is, the distance between the container 102 and the detector 106 increases. Sample 1 with 0% refined diesel shows the least fluorescence intensity ratios whereas Sample 6 with 50% refined diesel shows the largest fluorescence intensity ratios. In some implementations, the computer system 108 is configured to generate each straight line, for example, by implementing a line-fitting algorithm for the fluorescence intensity ratios at the five depths for each sample. In some implementations, the computer system 108 is also configured to display the five straight lines in the display device.

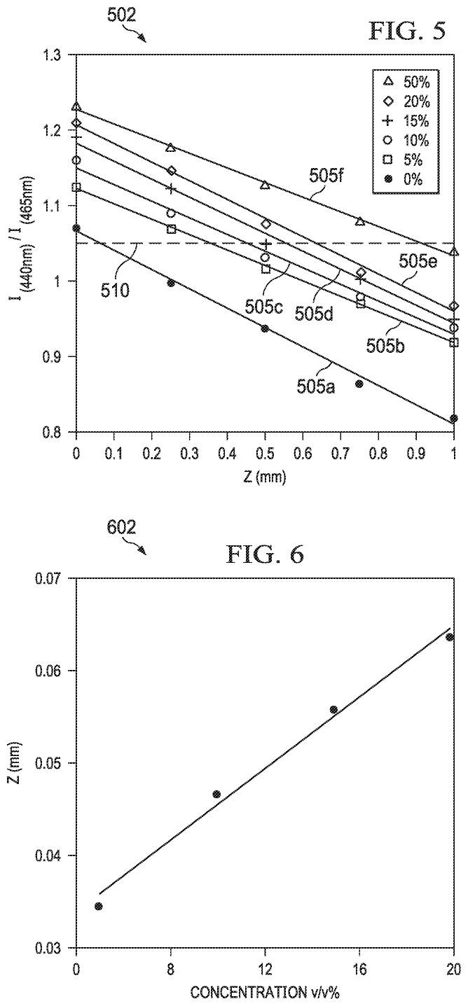

At 318, a calibration plot is constructed. For example, the computer system 108 is configured to construct the calibration plot from the plot 502 of the fluorescence intensity ratios versus depths. FIG. 6 is a calibration plot 602 generated from plot 502. To construct the calibration plot 602, the computer system 108 is configured to generate a horizontal line 510 (that is, a line parallel to the X-axis of plot 502) that intersects each straight line fitted for the fluorescence intensity ratios determined for the six samples. The horizontal line 510 represents a fluorescence intensity ratio that was determined for each depth of each sample. To determine the horizontal line 510, the computer system 108 can compare the fluorescence intensity ratios determined for all samples and identify a common fluorescence intensity ratio. The fluorescence intensity ratio is the ratio of two prominent peaks in the spectrum. Choosing the ratio based on the two prominent peaks allows taking advantage of the scattering effect. In the case of diesel as a sample, 440 nm can be chosen as that wavelength is characteristic for diesel and can be used to identify the presence of diesel in the hydrocarbon sample. Due to the scattering effect, this peak drops in intensity more than that of the peak at 465 nm, which lies on top of a background belonging to many hydrocarbons. In this manner, the choice of the ratio is sample specific. In the present example, the common fluorescence intensity ratio is 1.05. To construct the calibration plot 602, the computer system 108 can identify the depths that correspond to the intersection between the horizontal line 510 and the best-fit lines (505a-f), each of which corresponds to a particular % v/v value. The computer system 102 can generate the calibration plot 602 as a plot of the depths versus the % v/v values of the multiple samples. The computer system 108 can generate an equation that defines the plotted line. For the present example, the equation defining the horizontal line 510 is Y=0.00192X+0.026. Y is the depth; X is the % v/v value. The coefficient of determination for the horizontal line 510 is R₂=0.991.

Returning to FIG. 3, at 320, an unknown volume of hydrocarbon in an unknown hydrocarbon sample can be measured using the calibration plot. The unknown hydrocarbon sample is a sample that includes the same two hydrocarbons as those in the known hydrocarbon samples, for example, lubrication oil and refined diesel. However, the volume of the first hydrocarbon in the unknown hydrocarbon sample is unknown. To determine the unknown volume of the first hydrocarbon, the fluorescence spectra of the unknown hydrocarbon sample can be determined at different depths. Fluorescence intensity ratios can be determined at the different depths. At a certain depth, the fluorescence intensity ratio will correspond to the arbitrary fluorescence intensity ratio using which the calibration plot 602 was constructed. By substituting the determined depth into the equation for the calibration plot (that is, the horizontal line 510), the % v/v value for the unknown hydrocarbon sample can be determined. The determined % v/v value is the concentration of the first hydrocarbon in the unknown hydrocarbon sample. In some implementations, the computer system 108 can implement step 320 and provide an output, for example, display the % v/v value of the unknown hydrocarbon sample on the display device.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

1. A method comprising:
spatially positioning a container, an ultraviolet laser source and a detector relative to each other, the container carrying a hydrocarbon sample comprising a first hydrocarbon and a second hydrocarbon, the ultraviolet laser source configured to emit an ultraviolet laser at a wavelength to irradiate the hydrocarbon sample in the container, the wavelength configured to induce fluorescence in the hydrocarbon sample, the detector configured to detect the induced fluorescence;
irradiating the hydrocarbon sample in the container with the ultraviolet laser at a plurality of locations within the container at a respective plurality of distances from the detector, the respective plurality of locations arranged in a straight line normal to the detector; and
determining a volume of the first hydrocarbon in the hydrocarbon sample based on induced fluorescence detected by the detector at each of the plurality of locations arranged in the straight line normal to the detector.

2. The method of claim 1, wherein the induced fluorescence comprises a plot of fluorescence intensity over a range of wavelengths, wherein, the plot of fluorescence over the range of wavelengths at each of the plurality of locations comprises a greatest fluorescence intensity at a first wavelength of the range of wavelengths and a second greatest fluorescence intensity at a second wavelength of the range of wavelengths, wherein determining the volume of the first hydrocarbon comprises:
determining, at each of the plurality of locations, a respective greatest fluorescence intensity and a second greatest fluorescence intensity over the range of wavelengths; and
determining, at each of the plurality of locations, a fluorescence intensity ratio of the greatest fluorescence intensity and the second greatest fluorescence intensity.

3. The method of claim 2, wherein determining the volume of the first hydrocarbon further comprises constructing a calibration plot of the plurality of locations to a plurality of known volume ratios, each known volume ratio being a ratio of a known volume of the first hydrocarbon to a known volume of the hydrocarbon sample.

4. The method of claim 3, wherein the plurality of known volume ratios comprise six volume rations.

5. The method of claim 4, wherein the six volume ratios are 100%:0%, 95%:5%, 90%:10%, 85%:15%, 80%:20% and 50%:50%.

6. The method of claim 3, wherein constructing the calibration plot comprises:
preparing each known hydrocarbon sample by mixing a known volume of the first hydrocarbon with a known volume of the hydrocarbon sample resulting in a plurality of known hydrocarbon samples; and
for each known hydrocarbon sample:
placing a quantity of each known hydrocarbon sample in the container, and
irradiating each known hydrocarbon sample in the container with the ultraviolet laser at the plurality of locations within the container at the respective plurality of distances from the detector.

7. The method of claim 6, wherein constructing the calibration plot further comprises, for each known hydrocarbon sample:
measuring, at each location of the plurality of locations, a respective greatest fluorescence intensity and a second greatest fluorescence intensity over the range of wavelengths; and
determining, at each location of the plurality of locations, a fluorescence intensity ratio of the greatest fluorescence intensity and the second greatest fluorescence intensity resulting in a plurality of fluorescence intensity ratios at the respective plurality of locations for each known hydrocarbon sample.

8. The method of claim 7, wherein constructing the calibration plot further comprises:
constructing a plot of the plurality of fluorescence intensity ratios at the respective plurality of locations for the plurality of known hydrocarbon samples; and
identifying a known fluorescence intensity ratio that is the same at the plurality of locations for the plurality of known hydrocarbon samples as a reference fluorescence intensity ratio.

9. The method of claim 8, wherein determining the volume of the first hydrocarbon comprises determining, from the calibration plot, a location at which the fluorescence intensity ratio matches the reference fluorescence intensity ratio.

10. A system comprising:
a container configured to carry a hydrocarbon sample comprising a first hydrocarbon and a second hydrocarbon;
an ultraviolet laser source configured to emit an ultraviolet laser at a wavelength to irradiate the hydrocarbon sample in the container, the wavelength configured to induce fluorescence in the hydrocarbon sample; and
a detector configured to detect the induced fluorescence, the ultraviolet laser source positioned spatially relative to the container and the detector to irradiate the hydrocarbon sample in the container with the ultraviolet laser at a plurality of locations within the container at a respective plurality of distances from the detector, the respective plurality of locations arranged in a straight line normal to the detector; and
a computer system comprising:
one or more processors, and
a computer-readable medium storing instructions executable by the one or more processors to perform operations comprising determining a volume of the first hydrocarbon in the hydrocarbon sample based on induced fluorescence detected by the detector at each of the plurality of locations arranged in the straight line normal to the detector.

11. The system of claim 10, wherein the induced fluorescence comprises a plot of fluorescence intensity over a range of wavelengths, wherein, the plot of fluorescence over the range of wavelengths at each of the plurality of locations comprises a greatest fluorescence intensity at a first wavelength of the range of wavelengths and a second greatest fluorescence intensity at a second wavelength of the range of wavelengths, wherein determining the volume of the first hydrocarbon comprises:
determining, at each of the plurality of locations, a respective greatest fluorescence intensity and a second greatest fluorescence intensity over the range of wavelengths; and
determining, at each of the plurality of locations, a fluorescence intensity ratio of the greatest fluorescence intensity and the second greatest fluorescence intensity.

12. The system of claim 11, wherein determining the volume of the first hydrocarbon further comprises constructing a calibration plot of the plurality of locations to a plurality of known volume ratios, each known volume ratio being a ratio of a known volume of the first hydrocarbon to a known volume of the hydrocarbon sample.

13. The system of claim 12, wherein the plurality of known volume ratios comprise six volume ratios.

14. The system of claim 4, wherein the six volume ratios are 100%:0%, 95%:5%, 90%:10%, 85%:15%, 80%:20% and 50%:50%.

15. The system of claim 10, further comprising a motor configured to move the container to each of the plurality of distances from the detector.

16. The system of claim 10, wherein the detector is configured to perform operations comprising measuring, at each distance of the plurality of distances, a respective greatest fluorescence intensity and a second greatest fluorescence intensity over the range of wavelengths, and
wherein the operations performable by executing the instructions stored on the computer-readable medium comprise determining, at each distance of the plurality of distances, a fluorescence intensity ratio of the greatest fluorescence intensity and the second greatest fluorescence intensity resulting in a plurality of fluorescence intensity ratios at the respective plurality of locations for each known hydrocarbon sample.

17. The system of claim 10, wherein the ultraviolet laser source is positioned spatially relative to the container to direct the ultraviolet laser into the container in a first direction, and the detector is positioned spatially relative to the container to detect the induced fluorescence in a second direction that is perpendicular to the first direction.
